(19) United States
(12) Patent Application Publication
(10) Pub. No.: US 2012/0277239 A1
(43) Pub. Date: Nov. 1, 2012

(54) Compositions and Methods for Improving Night Vision

(75) Inventors: Gerald Horn, Deerfield, IL (US); Lee Nordan, Rancho Santa Fe, CA (US)

(73) Assignee: ALPHA SYNERGY DEVELOPMENT INC., Dana Point, CA (US)

(21) Appl. No.: 13/458,559

(22) Filed: Apr. 27, 2012

Related U.S. Application Data
(60) Provisional application No. 61/480,166, filed on Apr. 28, 2011.

(51) Int. Cl. A61K 31/498; A61P 27/14; A61P 27/02

(52) U.S. Cl. 514/249

(57) ABSTRACT

The invention provides compositions and methods for improving night vision without inducing redness or significant tachyphylaxis. The provided compositions and methods utilize low concentrations of selective α-2 adrenergic receptor agonists. The compositions preferably include brimonidine.

### CROSS-REFERENCE TO RELATED APPLICATION

This application claims priority of U.S. Provisional Application Ser. No. 61/480,166, filed Apr. 28, 2011, the disclosure of which is incorporated herein by reference.

### BACKGROUND OF THE INVENTION

Night vision refers to the ability to see in scotopic or mesopic (i.e., dim light or absent light) conditions. It is affected, among other factors, by pupil size. The average pupil diameter in normal light is between 6 mm to 7 mm, and the range of pupil diameter in dim light is typically 3 mm to 9 mm. Most disturbances in vision related to diminished light occur in individuals with mesopic pupils of 5.0 mm or greater. This is because the cornea is increasingly more optically imperfect when it is further from the corneal center; thus, optical imperfections or aberrations are typically greater in people with large pupils. However, any individual may experience problems with night vision, including difficulty with night driving, glare, halo, starburst, and/or reduced contrast.

Optical aberrations are typically measured in units of root mean square (RMS). Specific higher order aberrations which are more often associated with degradation of visual quality include spherical aberration (the most significant and common aberration), vertical coma and horizontal coma. However, other optical aberrations may also be clinically relevant.

Modulation of the pupil reaction to dim light allows for reduced light scatter in individuals with peripheral optical aberrations, which are estimated to include more than 30% of the population in the United States. The range of pupil dilation in the general population is largely genetically determined. For any specific pupil size, the amount of optical aberration is decreased when reduced dilation occurs relative to the genetic norm of the individual. Reduced dilation is especially clinically relevant for those with more significant or elevated optical aberrations. As long as the pupil size in reduced or absent lighting remains significantly above the 1 mm pupil (preferably, at least 2.5 mm, and more preferably 3 mm or greater), the reduction of optical aberrations not only does not cause diminution in acuity or peripheral vision, but improves vision quality in reduced or absent ambient light.

Contrast acuity, point spread function, or modulation transfer function (MLF) are means of measuring visual performance that relate to quantity and quality of vision, and which are affected by pupil size. This relationship to pupil size may be measured by varying light intensity, introduction of pharmacologic dilation or by introduction of pharmacologic reduction in pupil dilation to reduced light. For individuals with elevated RMS values, typically 0.5 RMS or greater, or with specifically elevated higher order aberrations, each increment of reduction in pupil dilation in response to dim light results in reduced light scatter, with less glare, and reduction in loss of contrast, halo, starburst, and other night vision visual problems.

The degree of visual improvement in terms of measured RMS units following pupil reduction in reduced vision is approximately proportional to pupil reduction, provided that the residual pupil diameter exceeds 2.5 mm, and preferably, 3 mm. The mean scotopic pupil in most populations is about 6.1 mm, with a range of about 4 mm to 9 mm. Most patients with night vision or mesopic vision issues will fall in a range of about 5.5 mm-9.0 mm pupils.

Thus, it may be desired to reduce pupil dilation in dim light vs. bright light via pharmacologic means. A pupil that increases from 1 mm, in bright sunlight to a maximum of only 3 mm still produces an an aperture allowing 900% more light than than the 1 mm pupil in bright sunlight, sufficient to allow near optimal mesopic and scotopic visual quality.

Conventional formulations used, or proposed to be used, to decrease pupil size have a number of drawbacks.

For example, pilocarpine, which is a direct acting miotic agent, causes pupil constriction as opposed to reduced dilation to dim light, thereby creates excessive dimness when first applied and the pupil in dim light is typically well below 3 mm, and frequently as small as 1 mm, is associated with brow ache, ciliary muscle contraction and pseudo myopia (muscle spasm of accommodation), and redness. Its effect lasts only a few hours, and it has a known risk of retinal detachment, probably related to pull on the retina from stimulated ciliary muscle contraction. For these reasons, it is rarely tolerated or considered a clinically useful alternative for patients with large pupils in dim light.

Another medication used to affect pupil size, an alpha-1 (α-1) adrenergic receptor antagonist dapiprazole (5,6,7,8-tetrahydro-3-[2-(4-o,tolyl-1-piperazinypethyl]-8-triazolo[4,3-a]pyridine hydrochloride), produces redness and frequently chemosis upon application and has very little effect on pupil size in dim light in clinical application.

Other α-1 antagonists, such as phentolamine, similarly cause surface vessel dilation via direct antagonism of α-1 agonists responsible for maintaining vascular tone and/or down-regulation of alpha receptors responsible for vasoconstriction, and thereby induce a loss of vasoconstrictive tone, which results in considerable hyperemia, which may lead to a cosmetic stigma, leakage, chemosis and significant discomfort and cosmesis issues.

α-1 antagonists are known to effectively reduce intraocular pressure and some have been commercialized for that purpose, for example, bunazosin. Bunazosin at 0.3% produces a pupil reduction of about 1.0 mm, an intraocular pressure reduction of about 20%, measured 6 hours post instillation with a mean conjunctival hyperemia score of 1.5/4, with a duration of about 24 hours before return to baseline. Trew et al, British Journal of Ophthalmology, 1991, 75, 411-413. Attempts to reduce hyperemia by administering topical vasoconstrictors, either pre-treatment or post-treatment, result in only transient improvement, lasting minutes to a few hours, which is significantly less than the duration of hyperemia

Some selective α-1 antagonists (such as sidolosin, KMD3213 and tamsulosin) are implicated in the “floppy iris syndrome” in which the pupil dilator muscle becomes atrophic, that is it has such an induced weakness (probably from disuse) that cataract surgery, when required, becomes a complex procedure with high morbidity and risk of prolapse of the iris into the anterior chamber with high risk of vitreous loss, retinal detachment, cystoid macular edema, vitreous wick syndrome, need for anterior and possibly posterior vitrectomy, and other related surgical complications. Oshika et al, Incidence of Intraopoertive Floppy Iris Syndrome in Patients on Either Systemic or Topical α1-Adrenoceptor Antagonist, Am J Ophthalmol 2007; 143:150-151.

Brimonidine at 0.10-0.20% has been used to create a reduced dilation of pupil in dim light, but results in nearly 100% of patients having total tachyphylaxis and loss of effect after several weeks of daily use. Brown et al, The effect of daily use of brimonidine tartrate on the dark-adapted pupil diameter., Am J Ophthalmol. 2004 July; 138(1); 149-151.I

Specifically, topically applied brimonidine at 0.15% results in about 25% of patients experiencing rebound hyperemia (redness), with 10% to 30% of the patients experiencing burning and stinging, blurring, foreign body sensation, conjunctival follicles, ocular allergic reactions and pruritis. 3% to 9% of the patients experience corneal, erosion, photophobia, eyelid erythema, ocular ache/pain, ocular dryness, tearing, eyelid edema, blepharitis, ocular irriation, and abnormal vision. At least 1% of the patients experience drowsiness or headaches.

Thus, there is a need for new compositions and methods that would improve night vision by achieving at least 0.10 mm reduction, and preferably equal to or greater than a 1.0 mm reduction in pupil size in reduced lighting conditions, with quick onset of ideally about 15 minutes or less, providing long term effective treatment for at least several weeks of continuous use at a time without tachyphylaxis, no induced eye redness, and preferably providing eye whitening to further improve compliance, without unintended intraocular pressure reduction and/or other side effects, and without requiring the use of α-1 general or selective antagonists.

### SUMMARY OF THE PRESENT INVENTION

The present invention provides compositions and methods for improving night vision which utilize compositions comprising low concentrations of selective alpha-2 (α-2) adrenergic receptor agonists.

The compositions and methods of the present invention do not require the use of α-1 general or selective antagonists and improve night vision with reduced or eliminated side effects, as compared to conventional methods.

In some embodiments of the invention, the selective α-2 adrenergic receptor agonists have binding affinities (Kᵢ) for α-2 over α-1 receptors of 500:1 or greater. In preferred embodiments of the invention, the selective α-2 adrenergic receptor agonists have Kᵢ for α-2 over α-1 receptors of 900:1 or greater, more preferably 1000:1 or greater, and most preferably, 1500:1 or greater.

It is a surprising discovery of the present invention that a selective α-2 adrenergic receptor agonist, when present in the formulation at a concentration from about 0.005% to about 0.05%; more preferably, from about 0.01% to about 0.03%, even more preferably, from about 0.02% to about 0.025% weight by volume of the composition, and when the formulation has pH of 6.7 or greater, preferably 7.0 or greater, and even more preferably 7.4 to 8.0, results in the pupil modulation which leads to an improvement of night vision. In preferred embodiments, a pH of the composition comprising a selective α-2 adrenergic receptor agonist is between about 7.0 and about 8.0.

The compositions which are at pH of 6.5 or less are not suitable for the purposes of the present invention because such modulation occurs too infrequently for commercial use, is more likely to be reduced in magnitude, and/or absent.

In preferred embodiments, the compositions and methods of the present invention provide a mean pupil reduction of about 20% average and 30% peak of the mesopic pupil diameter, or typically between 1.0 mm (in 5.0 mm mesopic pupils) and 2.0 mm or greater (in 8.0 mm mesopic pupils), without tachyphylaxis.

In preferred embodiments, the compositions and methods of the present invention further provide reducing eye redness and/or increasing eye whiteness.

In preferred embodiments of the invention, the selective α-2 adrenergic receptor agonist is selected from the group consisting of brimonidine, alpha methyl dopa, guanfacine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

It was surprisingly discovered that brimonidine at concentrations between 0.01% to 0.05% weight by volume, and particularly between 0.01% to 0.025% weight by volume, is compared to the equivalent concentration of oxymetazoline (a commercially used general α-agonist and vasoconstrictor) that brimonidine results in improved surface vasoconstriction, reduced surface redness, and improved whiter appearance.

The overall effectiveness of brimonidine is surprisingly found to be the best when pH is at about the pka value of brimonidine (pKa=7.69, ACD Labs) or within about 0.5 pH units (see, example 1). Thus, brimonidine is particularly effective for the purposes of the present invention at about neutral pH or above.

### DETAILED DESCRIPTION OF THE INVENTION

### Definitions

For purposes of the present invention, the terms below are defined as follows.

The term “selective α-2 adrenergic receptor agonists” encompasses all α-2 adrenergic receptor agonists which have a binding affinity of 100 fold or greater for α-2 over α-1 adrenergic receptors.

The term “low concentrations” refers to concentrations from between about 0.005% to about 0.05%; more preferably, from about 0.001% to about 0.03%; and even more preferably, from about 0.02% to about 0.025% weight by volume of the composition.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™, and UK14304.

As used herein, the term “composition” is intended to encompass a product comprising the specified ingredients in the specified amounts, as well as any product which results, directly or indirectly, from a combination of the specified ingredients in the specified amounts.

### Compositions and Methods of the Invention

At dim light conditions, a pupil naturally becomes dilated. Because the human cornea is optically imperfect, a significant number of people can poorly see at dim light conditions because their pupils, dilating larger than needed to allow in sufficient ambient dim light, introduce aberrant optical imperfections, which are frequently present in increased proportion to distance from the center of the cornea. Such patients will frequently notice improvement with as little as 0.2 mm of reduced dilation in dim light. This effect also may occur in other conditions when the pupil size increases beyond that associated with bright daylight, such as on cloudy days.

It: was surprisingly and unexpectedly found that selective α-2 adrenergic receptor agonists (which are interchangeably referred to as “α-2 agonists” throughout the application) at sufficiently low concentrations, and when formulated at: pH of 6.7 or greater, and more preferably 7.1 or greater, allow significant improvement in tissue hemodynamics and can be used for improving night vision. The use of such α-2 agonists at higher concentrations (as is typical for intraocular use) results in undesired downregulation and tachyphylaxis (loss of effect), as for example, with daily use of Alphagan® (brimonidine 0.15%) for four weeks as documented, for example, in Brown S M, Khanani A M, McCartney D L., The effect of daily use of brimonidine tartrate on the dark-adapted pupil diameter, Am J Ophthalmol. 2004 July; 138(1):149-51. It would take approximately six months of daily use of the compositions of the present invention to achieve the delivered dose equal to the one delivered by four weeks of daily use of 0.15% brimonidine.

The compositions and methods of the present invention allow achieving pupil modulation without the hyperemia caused by α-1 antagonists.

The invention provides compositions and methods for improving night vision utilizing low concentrations of selective α-2 agonists, including but not limited to, brimonidine and dexmedetomidine, when these compositions are at pH of 6.7 or above. The night vision is improved because the compositions used in the provided methods reduce pupil dilation at dim light conditions. Further, the methods of the present invention do not require the use of α-1 general or selective antagonists and result in reduced or eliminated side effects as compared to conventional methods.

In a preferred embodiment, the selective α-2 adrenergic receptor agonist is present at a concentration below about 0.05%, more preferably, between about 0.01% and about 0.03%, and even more preferably, between about 0.02% and about 0.025%; weight by volume of the composition.

The concentration of the selective α-2 adrenergic receptor agonist is sufficient to cause reduction in pupil dilation, yet below the concentration at which α-1 adrenergic receptors are sufficiently activated to cause adverse vasoconstrictive consequences, and it is prophetically predicted, below the dosing that causes downregulation and tachyphylaxis with daily use within weeks in many cases.

In preferred embodiments, the compositions and methods of the present invention further provide reducing eye redness and/or increasing eye whiteness.

In one embodiment, the selective α-2 adrenergic receptor agonist is selected from the group consisting of brimonidine, alpha methyl dopa, guanfacine, dexmedetomidine, fadolmidine, (+)-(S)-4-[1-(2,3-dimethyl-phenyl)-ethyl]-1,3-dihydro-imidazole-2-thione, 1-[(imidazolidin-2-yl)imino]indazole, and derivatives or mixtures of these compounds.

In a preferred embodiment, the composition comprises brimonidine at a concentration between about 0.01% and about 0.03% weight by volume.

In a more preferred embodiment, a pH of the composition comprising the selective α-2 adrenergic receptor agonist is between about 7.0 and about 8.0.

In one embodiment, the invention provides a method of improving night vision comprising administering to a patient in need thereof an aqueous composition formulated for a topical administration comprising between about 0.01% to about 0.03% weight by volume of brimonidine, wherein pH of said composition is between about 7.0 and about 8.0.

In another embodiment, the invention provides a method of improving night vision comprising administering to a patient in need thereof an aqueous composition comprising between about 0.01% to about 0.03% weight by volume of dexmedetomidine, wherein ,pH of said composition is between about 7.0 and about 8.0.

The compositions of the present invention are preferably formulated for a mammal, and more preferably, for a human.

In one embodiment, a pH of the compositions of the present invention is less than about 8.0, preferably, between about 7.0 and about 8.0, more preferably between about 7.4 and about 8.0.

The compositions and methods of the present invention can be used to optimize pupil size to obtain enhanced vision acuteness in dim light by reducing the pupil diameter in dim light, without substantially reducing the pupil size in bright light, when the pupil size does not need to be reduced to the same extent as the pupil under dim light.

In some embodiments, the optimized pupil diameter in dim light is no more than 200% greater than that in bright light, although preferably it is at 3 mm or greater. In some conditions, such as daylight associated with a cloudy day even slight increases in pupil size of magnitude less than 3 mm can reduce optical performance and benefit from pupil modulation.

While the compositions and methods of the present invention can be used to reduce naturally occurring pupillary dilation in dim light, especially in situations where the dilation is excessive to affect vision acuity, they can also be used also to counteract pupil dilation caused by medication.

Not wishing to be bound to a specific theory, it is believed that the methods and compositions of the present invention cause reduction in pupil dilation vias presynaptic dump of the catecholamine transmitters of the dilating nerve sympathetic innervation to the dilator muscles. This results in fewer transmitters available to trigger the pupil dilator muscle in dim light, which beneficially modulates (i.e., lessens) the otherwise natural increased pupil dilation in response to dim light. Not wishing to be bound to a specific theory, it is believed that the provided methods do not result in tachyphylaxis because of the significantly lower amounts of the α-2 agonist delivered to the eye as compared with conventional formulations.

It is a surprising discovery that using the very low concentrations as found to be effective for the present invention, and an α-2 agonist with low lipophilicity, such as brimonidine (Log P of only about 0.49), at pH of 6.7 or higher, or preferably 7.1 or higher, but not 6.5 or lower, allows to induce sufficient intraocular penetration to cause the desired pupil modulation in a majority of subjects. Thus, the α-2 agonist's penetration of the corneal epithelium is increased and therefore, the amount of the α-2 agonist which reaches the anterior chamber is also increased, allowing for extreme low doses of α-2 agonists to have substantial pupil modulation effect.

For example, compositions containing brimonidine at pH of 7.1 resulted in pupil modulation in 4 out of 4 individuals who administered topical brimonidine 0.025% at pH 7.1. See Example 1, below. One individual previously had used the same concentration at pH 6.5 with no pupil effect.

It is further believed that significant tachyphylaxis associated with brimonidine at 0.1%-0.2% is substantially reduced when the methods of the present inventions are employed.

In a preferred embodiment, the compositions and methods of the present invention provide a mean pupil reduction of about 2.0 mm without tachyphylaxis.

This is especially surprising since intraocular pressure reduction becomes marginal below 0.08%, and an estimated factor of concentration increase of about 10× is required to get drugs through the cornea. (Pharmacotherapy Perspectives, K. McGhie, Pharm D, Brimonidine, An alpha-2 adrenergic agonist for glaucoma; Journal of the Pharmacy Society of Wisconsin May/June 2001; p 2-36).

### Selective α-2 Adrenergic Receptor Agonists Suitable for the Purposes of the Invention

In some embodiments of the invention, selective α-2 adrenergic receptor agonists have binding affinities (Kᵢ) for α-2 over α-1 receptors of 500:1 or greater. In preferred embodiments of the invention, selective α-2 adrenergic receptor agonists have Kᵢ for α-2 over α-1 receptors of 900:1 or greater, more preferably 1000:1 or greater, and most preferably, 1500:1 or greater. Generally, a selective α-2 adrenergic receptor agonist which has Kᵢ for α-2 over α-1 receptors greater than that of oxymetazoline should be suitable for the purposes of the invention.

Not desiring to be bound by any specific theory or mechanism, it is believed that the particularly preferred adrenergic receptor agonists for the purposes of the present invention have higher selectivity for α-2A as compared to α-2B and/or α-2C receptors, receptors.

In preferred embodiments of the invention, concentrations of the selective α-2 adrenergic receptor agonists are from about 0.005% to about 0.05%; more preferably, from about 0.01% to about 0.03%; even more preferably, from about 0.02% to about 0.025% weight by volume of the composition.

Any selective α-2 adrenergic receptor agonist may be suitable for the purposes of the present invention. In one embodiment, the selective α-2 adrenergic receptor is selected from the group consisting of brimonidine, alpha methyl dopa, guanfacine, dexmedetomidine, fadolmidine, (+)-(S)-4-[1-(2,3-dimethyl-phenyl)-ethyl]-1,3-dihydro-imidazole-2-thione, 1-[(imidazolidin-2-yl)imino]indazole, and derivatives or mixtures of these compounds.

Compositions and methods of the inventions encompass all isomeric forms of the described α-2 adrenergic receptor agonists, their racemic mixtures, enol forms, solvated and, unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et: al., “Pharmaceutical Salts,” J. Pharm. Sci., 66: 1-19 (1977) which is incorporated herein by reference).

As long as a particular isomer, salt, analog, prodrug or other derivative of a selective α-2 adrenergic receptor agonist functions as a highly, selective α-2 agonist, it may be used for the purposes of the present invention.

When, choosing a particular α-2 adrenergic receptor agonist, one may take into account various considerations including blood brain permeability and any possible side effects and other systemic reactions.

In preferred embodiments of the invention, the selective α-2 adrenergic receptor is brimonidine or its salt. In a more preferred embodiment, the selective α-2 adrenergic receptor agonist is the tartrate salt of brimonidine.

### Methods of Administration

The compositions of the present invention may be applied topically to the eye, especially in the form of a solution, a suspension, an ointment, a gel or a solid insert. The dose of the active ingredient may depend on various factors, such as mode of administration, requirement, age and/or individual condition.

In one embodiment, the compositions of the invention may be placed on moist soft contact lens. The lens can then be inserted in the eyes for 15 to 45 minutes, once a day. The contact lens dosing allows for preferential absorption within the cornea, maximizing drop utilization.

### Additional Ingredients/Excipients

In some embodiments of the present invention, it may be necessary to improve (i.e., increase) the solubility of α-2 agonists. A greater solubility has a number of advantages, including but not limited to an ability to achieve higher concentrations and enhanced stability at storage at cold temperatures. Because the desired concentration of suitable α-2 agonists is very low, and the present invention provides formulations with much greater solubility, the desired concentrations are easily achieved even at an exponentially reduced known solubility in the desired near-alkaline to alkaline pH range.

For example, α-2 agonists, and more specifically, dexmedetomidine, are rendered more effective as well as more soluble by constituents of a balanced salt solution. The terms “salt” and “constituent of a balanced salt solution” are used interchangeably for the purposes of the present invention. They are a subset of agents that improve solubility of the inventive formulations.

Thus, in one embodiment of the present invention, dexmedetomidine is rendered soluble up to or beyond 0.1% at pH 7.1 by adding constituents of a balanced salt solution. In a preferred embodiment, these constituents include any combination of one or more of the following: sodium citrate dehydrate, sodium acetate, and calcium salt. In a more preferred embodiment, the concentration of sodium dehydrate is about 0.17%; the concentration of sodium acetate is about 0.39%; and the concentration of calcium salt is about 0.048%.

The most preferred agent that improves solubility is a citrate salt. Citrate salt acts as a preservative and a corneal penetration enhancer.

Other agents that improve solubility which may be used for the purposes of the present invention include, but are not limited to: methanesulfonate (mesylate), hydrobromide/bromide, acetate, fumarate, sulfate/bisulfate, succinate, citrate, phosphate, maleate, nitrate, tartrate, benzoate, carbonate, pamoate, borate, glycolate, pivylate, sodium citrate monohydrate, sodium citrate trihydrate, sodium carbonate, sodium EDTA, phosphoric acid, penatsodium pentetate, tetrasodium etidronate, tetrasodium pyrophosphate, diammonium ethylenediamine triacetate, hydroxyethyl-ethylenediamine triacetic acid, diethylenetriamine pentaacetic acid, nitriloacetic acid, and various other alkaline buffering salts, where polyanionic (multiple negatively charged) compounds, such as methylcellulose and derivatives, mucoadhesives, such as carboxymethyl cellulose (CMC), hydroxypropylmethyl cellulose (HPMC) or analogues and/or derivatives thereof, hyaluronic acid, and others may be particularly effective; and/or addition of cyclodextrins and/or their derivatives, particularly (2-Hydroxypropyl)-beta-cyclodextrin; certain solvents such as Tween 20, Tween 80, polyvinyl alcohol, propylene glycol and analogues or derivatives thereof; certain osmotic agents, such as mannitol or sucrose, HPMC, or certain chelating agents.

In preferred embodiments, concentrations of mucoadhesives are from about 0.05% to about 5% weight by volume, more preferably from about 1% to about 3%.

It is well within a skill of a skilled in the art to determine the amounts and concentrations of the additive agents.

In yet another preferred embodiment, the compositions of the present invention comprise nitrous oxide inhibitors. In a preferred embodiment, the nitrous oxide inhibitors are selected from the group consisting of L-NAME (L-N^{G}-Nitroarginine methyl ester), L-NIL (N6-(1-iminoethyl)-L-lysine dihydrochloride), L-NIO (N5-(1-Iminoethyl)-L-ornithine dihydrochloride), and L-canavine, or combinations thereof. Preferably, concentration of the nitrous oxide inhibitors is between about 0.005% and about 0.5% weight by volume.

The ophthalmic compositions may comprise further non-toxic excipients, such as emulsifiers, wetting agents or fillers, such as, for example, the polyethylene glycols designated 200, 300, 400 and 600, or Carbowax® designated 1000, 1500, 4000, 6000 and 10,000.

Other excipients that may be used if desired are listed below but they are not intended to limit in any way the scope of the possible excipients. They are especially complexing agents, such as disodium-EDTA or EDTA, antioxidants, such as ascorbic acid, acetylcysteine, cysteine, sodium hydrogen sulfite, butyl-hydroxyanisole, butyl-hydroxytoluene or α-tocopherol acetate; stabilizers, such as a cyclodextrin, thiourea, thiosorbitol, sodium dioctyl sulfosuccinate or monothioglycerol vitamin E and vitamin E derivatives, such as Vitamin E Tocopherol Polyethylene Glycol 1000 Succinate (TPGS); or other excipients, for example, lauric acid sorbitol ester, triethanol amine oleate or palmitic acid ester. The amount and type of excipient added is in accordance with the particular requirements and is generally in the range of from approximately 0.0001 to approximately 90% by weight.

The compositions may further include preservatives, which include, but are not limited to, benzalkonium chloride, chlorobutanol, thimerosal, phenylmercuric acetate, or phenylmercuric nitrate.

Delivery vehicles include, but are not limited to, polyvinyl alcohol, povidone, hydroxypropyl methyl cellulose, poloxamers, carboxymethyl cellulose, hydroxyethyl cellulose and purified water. It is also possible to use a physiological saline solution as a major vehicle.

Tonicity adjustors include, but are not limited to, a salt such as sodium chloride, potassium chloride, mannitol or glycerin, or another pharmaceutically or ophthalmically acceptable tonicity adjustor.

Buffers and pH adjustors include, but are not limited to, acetate buffers, citrate buffers, phosphate buffers and borate buffers. It is understood that acids or bases can be used to adjust the pH of the composition as needed.

Antioxidants include, but are not limited to, sodium metabisulfite, sodium thiosulfate, acetylcysteine, butylated hydroxyanisole and butylated hydroxytoluene.

To make the topical compositions of the present invention, one can simply dilute, using methods known in the art, more concentrated solutions of selective α-2 agonists. The precise method of carrying out the dilutions is not critical. Any commonly used diluents, including preservatives described above in the application, suitable for topical solutions can be used.

Proper dosages of the compositions of the present invention are concentration-dependent. To determine the specific dose for improving a night vision of a specific person, a skilled artisan would have to take into account kinetics and absorption characteristics of the particular selective α-2 adrenergic receptor agonist.

The following examples are provided solely for illustrative purposes and is not meant to limit the invention in any way.

### EXAMPLE 1

### Effect of Low Dose Brimonidine on Pupil Dilation

The purpose of this experiment was to test brimonidine at 0.025% on pupil dilation at two different pH, pH of 6.5 and pH of 7.1

### pH 6.5

Thirteen (13) individuals applied topical brimonidine 0.025%, pH 6.5, to one eye, using Blink® (a registered trademark of Abbott Medical Optics; active ingredient: polyethylene glycol 400 0.25%) artificial tear solution as a diluent for Alphagan P®, 0.15%.

Two (2) individuals experienced reduced dilation (appearing as miosis relative to the other eye) in modest indoor lighting conditions. This corresponds to 15.4% of the treated patients.

### pH 7.1

Four (4) individuals, all of which took part in the above-described experiment at pH of 6.5, were at a later date administered brimonidine 0.025%, pH 7.1 to one eye. All four experienced pupil modulation. This corresponds to 100% of the treated patients.

### EXAMPLE 2

### Effect of Low Dose Brimonidine on Pupil Dilation and IOP

The purpose of this experiment was to test brimonidine at 0.025% on pupil dilation at pH of 7.0.

Brimonidine 0.025%, pH 7.0 was applied to the right eye of a subject with baseline redness secondary to dry eye and a scotopic pupil of 5.0 mm in each eye, and the left eye as the contralateral control. All pupil measurements were made using Marco video monitor pupillometry in a darkened room, which was captured via digital image capture and monitor adjustable caliper measurement. Once the dark adapted pupil was digitially captured, the instrument allows superiposition of a caliper with 0.1 mm increments to measure the pupil diameter.

The obtained results are shown in Table 1:

TABLE 1

| | | Scotopic | | Scotopic | |
|---|---|---|---|---|---|
| | Redness | pupil size | | pupil size | |
| Time | (from 0 to 4) | (mm) | | (mm) | |
| (hr) | (right eye) | (right eye) | % reduction | (left eye) | % reduction |
| 0 | 2 | 5 | 0 | 5 | 0 |
| 0.05 | 0 | 5 | 0 | 5 | 0 |
| 0.5 | 0 | 4.5 | 10 | 5 | 0 |
| 1.25 | 0 | 4 | 20 | 5 | 0 |
| 2.25 | 0 | 3.5 | 30 | 4 | 20 |
| 3.25 | 0 | 3.5 | 30 | 4 | 20 |

Contralateral control (left eye) experienced a delayed but significant pupil modulation. Also, significant eye whitening was observed in the right eye at 0.5 hr, 1.25 hr, 2.25 hr and 3.25 hr.

### EXAMPLE 3

### Effect of Low Dose Brimonidine on Pupil Dilation and IOP

The purpose of this experiment was to test brimonidine at 0.025% on pupil dilation and IOP at pH of 7.0.

The subject was administered brimonidine 0.025%, pH 7.0 q am to one eye, recording the pupil size using a neuroptics pupillometer in dark conditions. The pupil size was measured at the baseline and then 10 minutes and 3 hours after instillation for 3 consecutive weeks.

### Results Summary:

Baseline: 6.2 mm (+/−about 0.2. mm)

### Day 1-20 Avg:

Onset: 10-15 min
1 hour: Mean of about 16%-20% reduction (1+ mm)
3 hours: Mean of about 30%-35% reduction (range about 3.8-4.2)

### Day 2.1 (follows 20 days of consecutive use).

No tachyphylaxis was noted.
Onset: 10-15 min
1 hour: Mean of about 16%-20% reduction (1+ mm)
3 hours: Mean of about 30%-35% reduction (range about 3.8-4.2)

What is claimed is:

1. A method of improving night vision in a patient in need thereof comprising administering to said patient a composition comprising a selective α-2 adrenergic receptor agonist having a binding affinity of 900 fold or greater for α-2 over α-1 adrenergic receptors, or a pharmaceutically acceptable salt thereof, wherein said selective α-2 agonist is at a concentration 0.06% or less weight by volume; and wherein pH of said composition is 6.7 or greater.

2. The method of claim 1, wherein said selective α-2 adrenergic receptor agonist is selected from the group consisting of brimonidine, alpha methyl dopa, guanfacine, dexmedetomidine, fadolmidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

3. The method of claim 1, wherein said selective α-2 adrenergic receptor agonist is brimonidine.

4. The method of claim 1, wherein said α-2 adrenergic receptor agonist is present at a concentration from between about 0.006% to about 0.06% weight by volume.

5. The method of claim 1, wherein said α-2 adrenergic receptor agonist is present at a concentration from between about 0.01% to about 0.03% weight by volume.

6. The method of claim 1, wherein pH of said composition is between 7.0 and 8.5.

7. The method of claim 1, wherein said method further comprises reducing eye redness and/or increasing eye whiteness in said patient.

8. A method of improving night vision in a patient in need thereof comprising administering to said patient a composition comprising brimonidine at a concentration of from about 0.005% to about 0.05% weight by volume, wherein said composition has a pH of between about 7.0 and about 8.5.

9. The method of claim 8, wherein said composition further comprises a balanced salt solution, wherein said balanced salt solution comprises sodium citrate salt and/or sodium citrate salt.

10. The method of claim 8, wherein said composition further comprises a chelator.

11. The method of claim 8, wherein said composition further comprises a cellulose derivative.

12. The method of claim 8, wherein said method substantially reduces or prevents tachyphylaxis, rebound congestion, and/or rebound mydriasis in said patient.

13. A topical composition for improving night vision comprising brimonidine at a concentration 0.05% or less weight by volume, and wherein pH of said composition is 6.7 or greater.

14. The topical composition of claim 13, wherein said concentration of brimonidine is between about 0.01% to about 0.03% weight by volume.

15. The topical composition of claim 13, wherein pH of said composition is between 7.0 and 8.0.

16. The topical composition of claim 13 wherein a preservative is selected from BAK, peroxide, or a peroxide derivative.
